(19) United States
(12) Patent Application Publication
(10) Pub. No.: US 2008/0073232 A1
(43) Pub. Date: Mar. 27, 2008

(54) System and method for reclaiming waste from diaper manufacture for the production of medical waste containers

(76) Inventors: Piyush J. Reshamwala, Crystal Lake, IL (US); John Japuntich, Harvard, IL (US)

Correspondence Address: RATNERPRESTIA, P O BOX 980, VALLEY FORGE, PA, 19482-0980, US

(21) Appl. No.: 11/901,339

(22) Filed: Sep. 17, 2007

Related U.S. Application Data
(60) Provisional application No. 60/846,292, filed on Sep. 21, 2006.

(51) Int. Cl. A61B 19/00; B29B 9/10

(52) U.S. Cl. 206/438; 264/37.33; 264/478

(57) ABSTRACT

A medical waste disposal container is made from reclaimed polypropylene in an amount of from greater than 0% to about 40% by weight and virgin polypropylene in an amount of from less than 100% to about 60% by weight. The medical waste disposal container has a puncture resistance of at least about 2.8 lbsf and an impact strength sufficient to prevent medical waste from escaping from the container. The method for forming medical waste disposal containers utilizing reclaimed plastic includes forming a supply of reclaimed pellets from film scraps of a first resin. The reclaimed pellets are combined with virgin pellets of a second resin to form a blend of pellets where the first resin is different from the second resin. The containers are formed by injection molding the blend of pellets.

### CROSS REFERENCE TO RELATED APPLICATIONS

This application claims the benefit of the filing date of the provisional application filed Sep. 21, 2006 and assigned Ser. No. 60/846,292, the contents of which are incorporated herein by reference.

### FIELD OF THE INVENTION

The invention is directed to a system and method for reclaiming plastic or resin, and more specifically, to a medical sharps waste disposal container and a method of manufacturing by injection molding a blend of a first resin reclaimed from a diaper manufacturing process and a second virgin resin.

### BACKGROUND OF THE INVENTION

The properties of plastics such as chemical resistance and durability make plastics an essential component in a wide variety of consumer-based products. However, these properties and the ubiquity of plastics present problems in connection with their disposal. For example, consumer-based disposable plastic products represent a large volume of non-biodegradable material in landfills. Direct recycling of a consumer-used plastic product for the manufacture of the same plastic product is not always feasible. Such consumer-used plastic is often contaminated with non-plastics, and the plastics that are present are often mixed with respect to polymer type. Separation and cleaning of the recycled plastic is typically uneconomical.

Pre-consumer plastics, i.e., plastic in the form of rejected parts, trim, and flash from manufacturing processes, represent a source of recyclable or reclaimable plastics. For example, U.S. Pat. No. 6,802,353 is directed to a process for recycling plastic sheets used in the process of making diapers. The process reclaims diaper trim by re-melting it with the same polymer of virgin resin material. This re-melt is then used to form a new plastic sheet in the same manufacturing process.

Because of the growing consumption of plastic products and the growing volume of plastic waste generated by the production of such products, there remains a need for improved systems for reclaiming waste.

### SUMMARY OF THE INVENTION

A exemplary method of forming containers of the present invention utilizes reclaimed resin. The method includes forming a supply of reclaimed pellets from a first resin. The reclaimed pellets are combined with pellets made from virgin resin of a different type to form a blend of pellets. The containers are formed by injection molding the blend of pellets.

Another exemplary method of manufacturing containers, more specifically, medical sharps waste disposal containers of the present invention include reclaiming plastic film scraps from a diaper manufacturing process. A supply of reclaimed pellets is formed from the plastic film scraps. The supply of reclaimed pellets is combined with pellets made from virgin polypropylene to form a blend of pellets. This blend of pellets is injection molded to form medical sharps waste disposal containers.

An exemplary medical sharps waste disposal container of the present invention is made from a blend of a first resin, for example, reclaimed polypropylene, which optionally includes trace amounts of other resins, for example a polyethylene. The first resin is in an amount of from greater than 0% to about 40% by weight, more preferably 0% to about 30% by weight, and a second resin, for example, virgin polypropylene, in an amount of from less than 100% to about 60% by weight, more preferably from less than about 100% to about 70% by weight. The container according to this exemplary embodiment has a puncture resistance of at least about 2.8 lbsf and an impact strength sufficient to prevent medical sharps disposed in the container from escaping from the container.

### BRIEF DESCRIPTION OF THE DRAWINGS

The invention is best understood from the following detailed description when read in connection with the accompanying drawings. It is emphasized that, according to common practice, the various features of the drawings are not to scale. On the contrary, the dimensions of the various features are arbitrarily expanded or reduced for clarity. Included in the drawings are the following figures:

FIG. 1 is a flow chart describing a method of forming a medical sharps waste disposal container according to an exemplary embodiment of the present invention;

FIG. 2 is a flow chart describing a method of forming a medical sharps waste disposal container according to another exemplary embodiment of the present invention; and

FIG. 3 is a medical sharps waste disposal container according to an exemplary embodiment of the present invention.

### DETAILED DESCRIPTION OF THE INVENTION

According to an exemplary embodiment, the present invention recycles plastic from a diaper-producing process for use in the manufacture of an injection molded plastic container. Specifically, the process of reclamation of diaper waste begins with the waste from a diaper forming process and ends with a sharps container for medical waste.

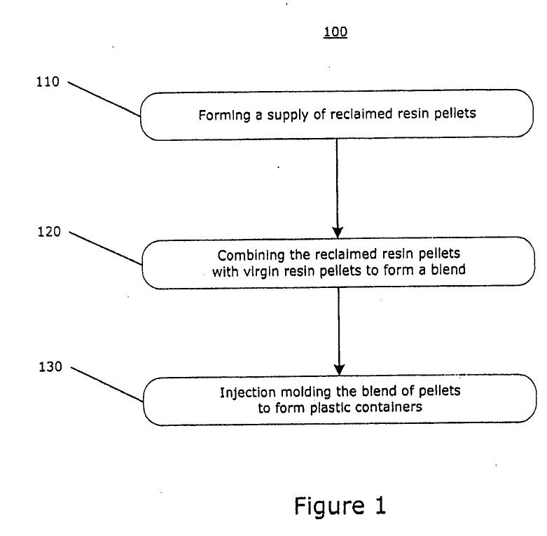

FIG. 1 is a flow chart describing method 100 of forming a medical sharps disposal container according to an exemplary embodiment of the present invention. Method 100 begins with block 110, forming a supply of reclaimed resin in the form of pellets. The reclaimed resin may be a sole polymer or a blend of polymers. According to an exemplary method, the forming step reclaims the resin from a production line of a product made from the reclaimed resin. The reclaimed pellets, shown by block 120, are combined with a pellets of a different polymer type to from a blend. Virgin resin is one example of a plastic of a different polymer type. Virgin resin is a term to describe a resin that has not be used in a manufacturing process of a plastic product or has otherwise been recycled or reclaimed. The blend of pellets, shown by block 130, is then injection molded to form plastic containers of the invention.

The step of forming the supply of reclaimed resin includes various intermediary steps. For example, when the production line is a diaper manufacturing process, one process of making diapers includes forming a sandwich of highly absorbent material between layers of resins, e.g., between layers of polypropylene. At a point in the manufacturing process, the legs holes of the diaper are cut from the polypropylene film portion of the sandwich. The leg cut-outs are typically semi-circular pieces of plastic film having a diameter of approximately 8 inches. Because this material is not contaminated during the diaper manufacturing process, it is considered a high quality film waste material. According to another embodiment, when the production line is a diaper manufacturing process, other waste material of another resin from other parts of the diaper are reclaimed. One such other waste material resin is polyethylene. Therefore, according to this exemplary embodiment, the step of forming the supply of reclaimed resin includes reclaiming a blend of resins, for example polypropylene and polyethylene. More preferably, the blend is predominantly polypropylene having minor, trace amounts of polyethylene.

Although the leg cut-outs are considered high quality film waste material, contamination may occur during transport and storage. The film waste may therefore need to be cleaned. An exemplary process to clean the high quality film waste material optionally includes shredding the film material into lengths. Suitable lengths include length less than 8 inches, lengths between about 4-8 inches, and lengths of 6-8 inches. After shredding, metal contaminates are removed by using any customary process as would be understood by one of ordinary skill in the art. The metal-free film is shredded into smaller fragments known in the industry as fines. This shredding process that form the fines frictionally pre-heats the fines to over about 100° F., for example to between about 100° F. and 150° F. The fines are then melted into a flowable material. This flowable material can be further cleaned by homogenizing the flowable material. Homogenizing the flowable material includes passing the flowable material through a filter or screen to remove final contaminates. The filter or screen has a size of about 40 to about 80 mesh, although other mesh sizes could be used as well.

After the high quality film waste material is cleaned and or homogenized, it can be formed into reclaimed pellets. One exemplary method of forming reclaimed pellets includes extruding the high quality film waste material in its flowable form through a multi-orifice die to form strands. The strands are cooled and cut into manageable sized pellets. In this manner, a supply of reclaimed pellets from high quality film waste material is formed.

The supply of reclaimed pellets from high quality film waste material (i.e., a first resin) is combined with pellets of a second resin, (e.g., either reclaimed or virgin resin material) to form a blend of pellets. The blend of pellets contains the first resin in an amount of from greater than 0% to about 40% by weight, more preferably from about 0% to about 30% by weight, and the second resin in an amount of from less than 100% to about 60% by weight, more preferably from less than about 100% to about 70% by weight. According to one exemplary embodiment, the first resin is polypropylene which optionally has trace amounts of polyethylene and the second resin is polypropylene. According to another exemplary embodiment, the polypropylene is reclaimed from a production line process, for example a diaper production line process, and the polypropylene is virgin resin material.

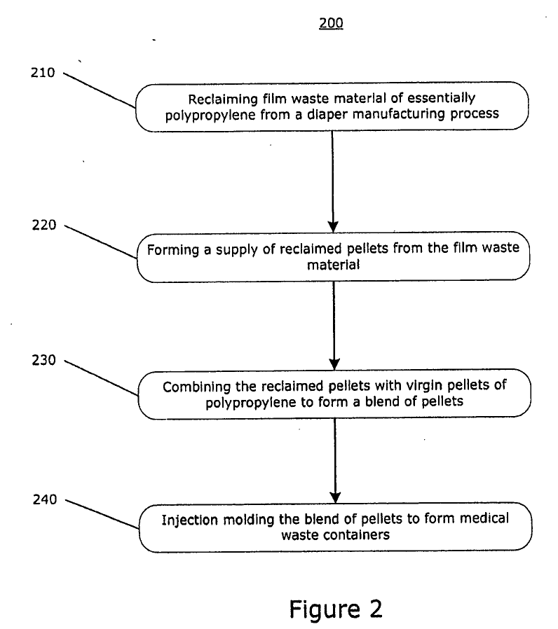

FIG. 2 is a flow chart describing method 200 of forming a medical sharps waste disposal container according to another exemplary embodiment of the present invention. Method 200 begins with block 210, which includes the step of reclaiming film scraps of polypropylene from plastic film used in a diaper manufacturing process. The scraps are formed into a supply of reclaimed pellets as shown in block 220. In block 230, the reclaimed pellets are combined with virgin polypropylene pellets to form a blend of pellets. The blend of pellets, as shown by block 240, is injection molded to form exemplary containers, such as medical waste sharps disposal containers.

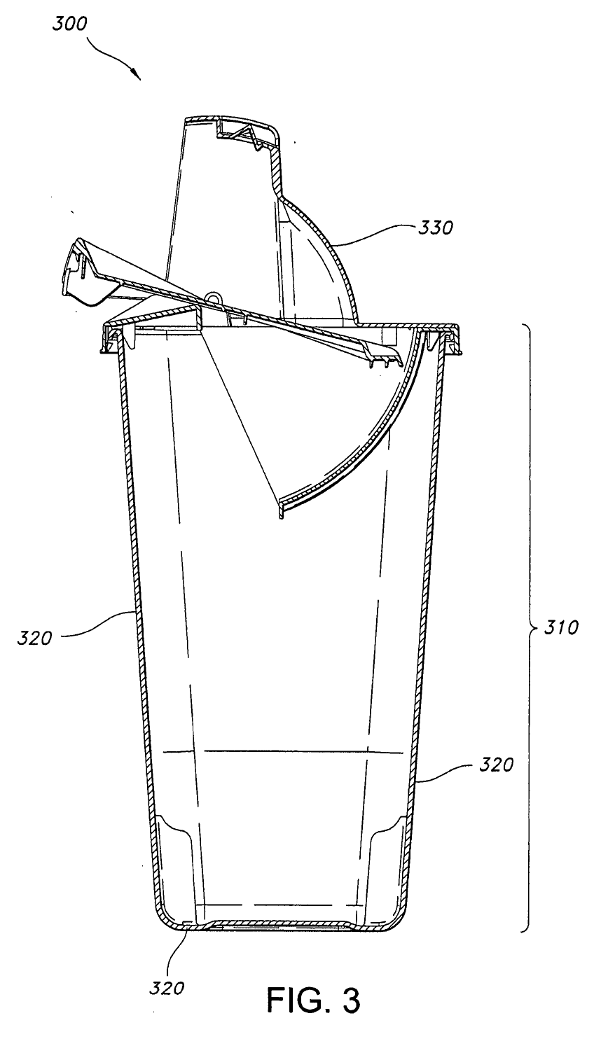

An exemplary container of the present invention is medical waste sharps disposal container 300, illustrated in FIG. 3. Medical sharps waste disposal container 300 has a base 310 made from a plurality of walls 320 and a top 330. Base 310 is of a unibody design or comprises separate components. According to an exemplary embodiment, base 310 and walls 320 are made from a first resin, for example, reclaimed polypropylene, which optionally has trace amounts of polyethylene, from a diaper manufacturing process, in an amount of from greater than 0% to about 40% by weight, for example about 30% by weight. The first resin is combined with a second resin, for example, virgin polypropylene, in an amount of from less than 100% to about 60% by weight for example about 70% by weight. The medical sharps waste disposal container has a suitable impact strength sufficient to prevent medical waste from escaping from the container if the container is dropped and a suitable puncture resistance to prevent medical sharps from puncturing the base and walls of the container. According to one embodiment, a container of the present invention has a puncture resistance of at least about 2.8 lbf. Alternatively, the container has an average puncture resistance of at least about 3.4 lbf, or at least 5.0 lbf.

ASTM-F2132 provides a test procedure and performance requirement for the puncture resistance of materials used in the construction of containers for discarded medical waste, needles and other sharps. This test specification establishes (1) the average puncture force and (2) a minimum value of puncture force that container materials must withstand when following the test procedure. According to one exemplary embodiment, the medical sharps and waste disposal container of the present invention has an average puncture resistance of at least about 3.4 lbf., preferably at least about 5.0 lbf., wherein the minimum requirement is preferably at least about 2.8 lbf.

Another structural characteristic is impact strength. A test procedure that measures impact strength is ASTM-D5628, which determines the relative ranking of materials according to the energy required to crack or break flat, rigid plastic specimens under various specified conditions of impact of a free-falling dart. Another test for impact strength is to drop a filled, medical sharps and waste disposal container from a predetermined height (the height depends on the size and weight of the container) onto a hard surface. The container fails this impact strength test when the impact of the drop causes a medical sharp or other medical waste to escape from the container. For example, a filled, 2 gallon medical sharps disposal container weighing about 1.0 lbs was dropped from a height of 36 inches. If no medical sharps or medical waste escaped from the container, either through a breach in a wall or the lid of the container, after being drop from the predetermined height, the container is determined to have a sufficient impact strength.

Although the invention is illustrated and described herein with reference to specific embodiments, the invention is not intended to be limited to the details shown. Rather, various modifications may be made in the details within the scope and range of equivalents of the claims and without departing from the invention.

What is claimed:

1. A method of forming containers utilizing reclaimed resin, the method comprising the steps of:
forming reclaimed pellets from film scraps of a first resin;
combining the reclaimed pellets with virgin pellets of a second resin to form a blend of pellets, the first resin being different from the second resin; and
injection molding the blend of pellets to form containers.

2. The method of claim 1, wherein the forming step comprises reclaiming the first resin from a production line of a product made from the first resin.

3. The method of claim 2, wherein the production line is a diaper manufacturing process.

4. The method of claim 1, wherein the forming step comprises the steps of:
shredding the film scraps of the first resin;
removing metallic contaminates from the film scraps of the first resin;
shredding the film scraps of the first resin into fines, which shredding process frictionally pre-heats the fines to approximately 100° to 150° F.; and
melting the fines into a flowable material.

5. The method of claim 4, wherein the shredding step shreds the film into 6-8 inch lengths.

6. The method of claim 1, wherein the forming step further comprises the steps of:
passing a flowable material made from the film scraps of the first resin through a particulate screen to remove contaminates;
extruding the flowable material through a multi-orifice die to form strands; and
cutting the strands to form pellets.

7. The method of claim 1, wherein the blend of pellets comprises the first resin in an amount of from greater than 0% to about 40% by weight and the second resin in an amount of from less than 100% to about 60% by weight.

8. The method of claim 7, wherein the first resin comprises reclaimed polypropylene and the second resin comprises virgin polypropylene.

9. The method of claim 1, wherein the injection molding step forms a medical waste disposal container.

10. The method of claim 9, wherein the medical waste disposal container has a puncture resistance of at least about 2.8 lbf and an impact strength sufficient to prevent medical waste from escaping from the container.

11. A medical waste disposal container comprising:
reclaimed polypropylene in an amount of from greater than 0% to about 40% by weight; and
virgin polypropylene in an amount of from less than 100% to about 60% by weight;
the medical waste disposal container having a puncture resistance of at least about 2.8 lbf and an impact strength sufficient to prevent medical waste from escaping from the container.

12. The medical waste disposal container of claim 11, wherein the reclaimed polypropylene comprises film scraps from a diaper manufacturing process.

13. The medical waste disposal container of claim 11, wherein the container has an average puncture resistance of at least about 3.4 lbf.

14. The medical waste disposal container of claim 11, wherein the container has an average puncture resistance of at least about 5.0 lbf.

15. The medical waste disposal container of claim 11, wherein the virgin polypropylene comprises about 70% by total weight of the container.

16. The medical waste disposal container of claim 11, wherein the reclaimed polypropylene comprises about 30% by total weight of the container.

17. A method of manufacturing medical waste containers, the method comprising the steps of:
reclaiming film scraps of polypropylene from a diaper manufacturing process;
forming reclaimed pellets from the film scraps;
combining the reclaimed pellets with virgin pellets of polypropylene to form a blend of pellets; and
injection molding the blend of pellets to form medical waste containers.

18. The method of claim 17, wherein the reclaiming step comprises shredding the polypropylene film scraps;
removing metallic contaminates from the polypropylene film scraps;
shredding the polypropylene film scraps of the first plastic into fines, which shredding process frictionally pre-heats the fines to approximately 100° to 150° F.; and
melting the fines into a flowable material.

19. The method of claim 18, wherein the forming step comprises the steps of
passing a flowable material made from the reclaimed film scraps of polypropylene through a particulate screen to remove contaminates;
extruding the flowable material through a multi-orifice die to form rods; and
cutting the rods to form pellets.
